(19) United States
(12) Patent Application Publication
(10) Pub. No.: US 2013/0203844 A1
(43) Pub. Date: Aug. 8, 2013

(54) Method for Stimulating the Reproductive Capacity in a Bull, and Composition for Stimulating the Reproductive Capacity in a Bull

(71) Applicants: Patricia Meillour, Lille (FR); Chrystelle Le Danvic, Lille (FR); Patrice Humblot, St. Maur (FR); Philippe Chemineau, Vouvray (FR); Christine Briant, Villedomer (FR); Olivier Gerard, Blain (DE)

(72) Inventors: Patricia Meillour, Lille (FR); Chrystelle Le Danvic, Lille (FR); Patrice Humblot, St. Maur (FR); Philippe Chemineau, Vouvray (FR); Christine Briant, Villedomer (FR); Olivier Gerard, Blain (DE)

(21) Appl. No.: 13/636,002

(22) PCT Filed: Mar. 28, 2011
(86) PCT No.: PCT/EP2011/054747

§ 371 Date: Apr. 24, 2013

(30) Foreign Application Priority Data
Mar. 30, 2010 (FR) 1052329

(51) Int. Cl. A61K 31/035; A61K 31/201; A61K 31/131; A61K 31/121; A61K 31/37; A61K 31/01

(52) U.S. Cl. CPC A61K 31/035; A61K 31/37; A61K 31/01; A61K 31/131; A61K 31/121; A61K 31/201
USPC 514/457; 514/762; 514/663; 514/675; 514/560; 514/744

(57) ABSTRACT

The invention relates to the use of a composition comprising at least one volatile molecule specific to the estrus of cows in order to improve the reproductive function of a bull. In particular, the composition according to the invention makes it possible to act on the libido of the bull, but also on the production of semen by the bull. Preferably, the composition used comprises at least one molecule from among coumarin, squalene, 6-amino undecane, 2-butanone, 9-octadecenoic acid and 1,2-dichloroethylene.

### FIELD OF THE INVENTION

The invention relates to a method for stimulating the reproductive capacity in a bull. More specifically, the method according to the invention makes it possible to stimulate the sexual function, in particular the libido, of the bull, and also improve the quality of the semen. The invention also relates to a composition intended for use on bulls with a view to stimulating their reproductive capacity.

The invention finds applications in the field of cattle breeding and, in particular, for collecting semen from bulls and inseminating cows, directly or artificially.

### STATE OF THE ART AND PROBLEMS ENCOUNTERED

Recent studies have shown that the estrus stage, corresponding to the period during which the female is receptive to males and can be fertilized, is especially short in cows, about 6 to 12 hours. During the estrus phase, a cow secretes volatile sexual pheromones informing the males of her availability for mating. Thus, several studies have been carried out on cattle in order to confirm this hypothesis. These studies have made it possible to confirm the presence of such molecules in several secretions, and notably in the vaginal secretions and urine, and their action on the Flehmen of the males. These molecules are detected by the males and allow them to identify females in the estrus stage.

Currently, cattle are most often selected according to specific characteristics, related to the goal of the breeding. Thus cows from dairy breeds are selected according to their lactation capacity so as to ensure a constant milk production, in terms of both quality and quantity. Similarly, beef breeds are selected for the quantitative, nutritional, taste, etc. qualities of their meat. Bovine reproduction is therefore rarely left to chance; both bulls and cows are selected according to specific criteria related to the final destination of the offspring.

Collecting the semen of bulls in a semen production center is a delicate operation that must be carried out rigorously not only with regard to the collection itself, in particular to prevent contamination, but also when the bulls are handled, before and after the collection.

Currently it is not possible to develop a protocol common to all of a farm's bulls so as to ensure an almost uniform collection among all the farm's individuals. In effect, individuals of the same farm may have a variable libido, both from one individual to the next and, for a single individual, from one moment to the next.

To date, there is no effective method to stimulate the sexual function and, more particularly, the libido of the bulls used in an insemination center or on a farm. However, libido problems are extremely common among bulls and are prejudicial for semen selection centers. They can result in not insignificant economic losses (loss of doses, significant loss of time, risks for the drovers). The time and conditions of collection are therefore difficult parameters for the drovers to predict.

Similarly, the quality and quantity of the semen fluctuate from one individual to the next and, for a single individual, from one moment to the next. To date, there is no effective method to improve the production of semen by bulls other than the individualized management of collections, taking each individual's behavior into account.

### DESCRIPTION OF THE INVENTION

The purpose of the invention is to overcome all or part of the problems described above. In particular, one of the aims of the invention is to improve the reproductive capacity of bulls in order to collect their semen for subsequent inseminations and/or for mating with females in estrus stage. ‘Reproductive capacity’ means behavioral capacity, in particular the libido, and/or physiological capacity, in particular the quality and/or quantity of the semen.

To achieve this, the invention proposes to use certain molecules, initially isolated from certain secretions of cows in the estrus stage, and in particular in the urine, for presenting to bulls before collecting their semen or before presenting the bulls to females for reproduction. In particular the inventors have identified six molecules specific to the estrus stage that have effects on the reproductive capacity of bulls, namely coumarin, squalene, 6-amino undecane, 2-butanone, 9-octadecenoic acid and 1,2-dichloroethylene.

These molecules can be isolated from secretions of cows in the estrus stage or be obtained by any other means, in particular chemical synthesis. Moreover, some of these molecules are already available on the market.

The molecules according to the invention are both releaser pheromones, i.e. pheromones that act on the behavior, and also primer pheromones, i.e. pheromones that act on the physiology. More specifically, inhaling these molecules makes it possible to reduce the time before mounting and ejaculation and/or increase the spermatozoa concentration in the ejaculate.

The uses made of these enable, with an equivalent environmental management of collection, semen production to be increased for all the individuals, with negligible treatment/individual interaction effects. This improvement in the semen production is very interesting in economic terms, in particular for individuals with a high genetic value. In addition, obtaining similar effects among young animals is very interesting, especially in the context of genomic selection where optimizing the reproductive performance of such individuals is sought in order to accelerate genetic progress.

More particularly, the invention proposes to use these molecules in a solution able to be sprayed into the nostrils of the bulls to be treated. In effect, in this way the molecules are in direct contact with the target and the risks of the molecules, which are very volatile, dissipating in the air are reduced. In addition, this mode of administration means that the quantity of pheromones inhaled by each individual can be precisely dosed.

### BRIEF DESCRIPTION OF THE FIGURES

FIG. 1 shows the comparative chromatographic profiles of the chemical composition of the cow's urine during an estrous cycle;

FIGS. 2A to 2F show the experimental ion spectra (a), compared with the spectra of the NIST database (b), of six chemical compounds specific to the pre-estrus and estrus stages, identified according to the invention, in the urine and vaginal mucus of cows;

FIGS. 3A to 3C show the effects of a first example of treatment according to the invention on the libido (FIG. 3A), spermatozoa concentration of the ejaculates in billion spermatozoa/mL (FIG. 3B) and on the total number of spermatozoa in billions, per ejaculate (FIG. 3C);

FIGS. 4A and 4B show the effects of the first example of treatment according to the invention on the semen of a group of bulls with normal libido (spermatozoa concentration: FIG. 4A; total number of spermatozoa: FIG. 4B);

FIG. 5 shows the effects of the first example of treatment according to the invention on the libido of a group of bulls with low initial libido;

FIGS. 6A and 6B show the effects of a second example of treatment according to the invention on the libido (FIG. 6A) and semen (FIG. 6B) of bulls, according to their initial libido.

### DETAILED DESCRIPTION OF THE INVENTION

The inventors have identified volatile molecules, specifically present in some secretions of cows in estrus stage, and that have a beneficial effect on the reproductive function of bulls, in that they are able to improve and/or induce the libido of bulls, and/or to improve the quality and/or quantity of semen produced.

According to the invention, the sexual behavior, or libido, of bulls takes into account the reaction time, the sexual preparation time and the time to ejaculation. Reaction time is defined according to the invention as the interval between the time the bull is brought to about 1.5 m from the teaser and the first false mounting. Sexual preparation time corresponds to the preparation recommended by Amann (1976), namely a false mounting, followed by 2 minutes of active waiting, then two false mountings at the bull's rhythm, and finally, without further waiting, the mounting leading to ejaculation. Active waiting is defined by the bull being kept in contact with the teaser, the two animals being side by side. Time to ejaculation is equal to the sum of the reaction time and the sexual preparation time.

The object of the invention is therefore the use of a composition comprising at least one volatile molecule specific to the estrus of cows to improve reproductive function in a bull.

‘Cow’ means the female of the bovine species of reproductive age, i.e. most often over 18 months old. Similarly, ‘bull’ means the male of the bovine species, not castrated and of reproductive age.

Estrus corresponds to the period of the estrous cycle, also called heat, during which ovulation takes place and the female accepts mating. Estrus is preceded by the pre-estrus phase and followed by the post-estrus phase.

‘Reproductive function’ means the behavioral and physiological processes leading to the production of semen. According to the invention, the improvement in reproductive function may consist of an improvement in libido, i.e. an improvement in the behavior leading to the production of semen, of an improvement in the semen itself, or of both.

‘Improvement’ means an increase in the bull's reproductive capacity, whether by reducing the time required for said bull to produce semen and/or by increasing the intrinsic quality of the semen itself. In particular, the quality of the semen can be improved by increasing the volume produced, increasing the concentration of spermatozoa in said volume, increasing the number of motile spermatozoa, etc.

According to the invention, the use of this composition makes it possible to improve the performance of the semen collection for the bull treated, or increase the chances of obtaining fertilization after the treated bull has served a cow in heat.

Preferably, in the composition used at least one volatile molecule specific to the estrus stage of cows is selected from among coumarin, squalene, 6-amino undecane, 2-butanone, 9-octadecenoic acid and 1,2-dichloroethylene. Advantageously, the composition contains at least a mixture of coumarin and squalene.

In other preferred modes of implementation of the invention, the composition comprises at least a mixture of squalene and 1,2-dichloroethylene. Any combination of two or more of the above volatile molecules specific to the estrus stage of cows also falls within the invention.

The composition according to the invention is advantageously presented in the form of a solution comprising glycerol as a solvent of the volatile molecules.

Preferably, the total concentration of volatile molecules specific to the estrus stage is between 2.5 pg/mL and 500 pg/mL. In particular, it is between 5 pg/mL and 100 pg/mL, or between 2.5 pg/mL and 5 pg/mL, or between 100 pg/mL and 500 pg/mL. ‘Total concentration’ means the sum of the concentrations of each of the volatile molecules specific to estrus stage used to produce the composition. Even more preferably, the concentration of volatile molecules specific to estrus in the composition is at least equal to 25 pg/mL, +/−5%.

In other preferred embodiments of the invention, the concentration of each volatile molecule specific to the estrus stage of cows contained in the composition is greater than or equal to 2.5 pg/mL.

According to examples of use of the composition according to the invention, it is possible to keep said composition at the periphery of the bull's nostrils so that said bull can inhale said composition.

Preferably, the composition is presented in spray form so it can be sprayed directly into the bull's nostrils.

According to the invention, the composition can advantageously be used with immediate effect. In other words, there is no latency time for the composition to act and covering a female or collecting the semen can take place directly after said composition has been presented to the bull.

Advantageously, said composition is presented to the bull before each semen collection or before each cow covering.

The invention also relates to a composition designed for use on a bull, in order to improve its reproductive function, characterized in that it comprises at least one volatile molecule specific to the estrus of cows.

Advantageously, in the composition at least one volatile molecule specific to the estrus of cows is selected from among coumarin, squalene, 6-amino undecane, 2-butanone, 9-octadecenoic acid and 1,2-dichloroethylene.

In other preferred embodiments of the invention the composition comprises at least a mixture of squalene and 1,2-dichloroethylene. Any combination of two or more of the above volatile molecules specific to the estrus stage of cows also falls within the framework of the invention.

The composition can be presented in the form of a solution preferably containing glycerol as the solvent of the volatile molecules. In effect, glycerol is not harmful to the animal and has no action on the bull's reproductive function. Of course, any other solvent or excipient able to dilute the molecules according to the invention, and enabling use with no risk to the bull, can be used.

Preferably, the solution comprises the excipients necessary to enable it to be sprayed.

According to the invention, the solution advantageously has a total concentration of volatile molecules specific to the estrus stage of between 2.5 pg/mL and 500 pg/mL. In particular, it is between 5 pg/mL and 100 pg/mL, or between 2.5 pg/mL and 5 pg/mL, or between 100 pg/mL and 500 pg/mL. Even more preferably, the concentration of volatile molecules specific to the estrus in the composition is at least equal to 25 pg/mL, +/−5%.

In other preferred embodiments of the invention, the concentration of each volatile molecule specific to the estrus stage of cows contained in the composition is greater than or equal to 2.5 pg/mL.

Experiments

I—Materials and Methods

### 1—Collection of Biological Samples (Urine and Vaginal Mucus)

In order to identify the chemical compounds involved in the detection of estrus by male cattle, several collections of urine and vaginal mucus were carried out on cull cows or heifers.

In a first step, two series of collections were carried out on Prim'Holstein cull cows.

Urine was collected from animals synchronized by treatment with prostaglandin (PGF2α) at the time of natural micturition at three stages in the cycle, namely pre-estrus (D+1 post-PGF2α), estrus (D+2 post-PGF2α) and in the luteal phase (D+7 post-PGF2α). The cycles were monitored during these collections, so as to better determine the time of estrus, by daily observation of the animals combined with measuring plasma progesterone levels.

Vaginal mucus was also collected by swabbing with gauze on the peri-vaginal area when vaginal mucus occurred.

A collection session was also performed withl8-month heifers during their first estrous cycles. Urine samples were collected in the pre-estrus (D-3 to D-1 before estrus), estrus and luteal (D+10) phases during natural cycles. The stages were determined by carefully monitoring behavior through 5 15-minute visual observations per day over more than 36 months. These were combined with ultrasound scans, 3 times a week, to monitor follicular maturation.

Three other collection sessions were carried out as follows: Six to eight cows (Holstein and Montbeliard) were synchronized in their cycle by two injections of prostaglandin (PGF2a) 10 days apart, with a second injection on the day of the first collections (D0). Plasma hormone levels were measured in addition to visual and behavioral observations in order to determine the time of ovulation. Blood samples were thus taken every 6 hours in order to measure levels of luteinizing hormone (LH) and estradiol (E2) during the week following the second injection of prostaglandin. Progesterone (P4) levels were measured every 24 hours. The serum from each blood sample was stored at −80° C. until the measure of the levels of progesterone (ovucheck plasma kit, Biovet), luteinizing hormone (LH DETECT kit, INRA, Nouzilly) and estradiol. Follicular development was monitored in addition, during one of the collections, through two ultrasound scans per day.

Urine was collected during spontaneous micturition two to three times a day depending on the animals. At the time of the collection, the urine was divided into 4 4-ml glass tubes sealed with Teflon stoppers (Supelco).

Cervical mucus, biological fluids specific to estrus conditions, was also collected in 20-ml glass bottles closed by Teflon stoppers (Supelco).

In all cases, the samples collected were stored at 4° C. after collection before storage within 4 hours at −80° C. until they were analyzed.

### 2—Analysis of Chemical Composition of the Biological Fluids Sampled

2-1—Extraction by Organic Solvent

The chemical extraction was performed volume to volume with an organic solvent from 4 mL of urine or vaginal mucus thawed on ice.

Several solvents were tested (dichloromethane (DCM), pentane, hexane). DCM, which showed a capacity for extracting a larger number of chemical compounds on the first samples analyzed, was selected. Extractions were made in glass tubes closed with Teflon screw caps (Supelco) to minimize contamination by plastic residues (phthalates).

After a quick vortex, samples were sedimented at ambient temperature for 10 min before being centrifuged at 2500 rpm for 20 min. After this centrifugation, the organic phase was dried over a sodium sulfate column (FLUKA) before being concentrated 100 times under a stream of nitrogen and stored at −20° C. until analysis (within two weeks following the extraction).

2-2—Identification of Chemical Compounds by GC/MS

The chemical extracts were analyzed by gas chromatography combined with mass spectrometry (GC-MS) on a FOCUS/DSQII (Thermo Scientific) apparatus consisting of a Focus GC coupled to a quadrupole mass spectrometer (DSQII, Thermo Scientific).

During the analyses (conducted over 3 years), five different columns were used to separate the extracted compounds: a CP-sil5 CB low Bleed/MS 60 m×0.25 μm ID×0.25 μm (SUPELCO); a TR-1MS 30 m×0.25 mm ID×0.25 μm (Thermo); a 1MS-SolGel 30 m×0.25 μm ID×0.25 μm (SGE); a TR-1MS 30 m×0.25 ID×0.25 m (Thermo); a TR-1MS 30m×0.25 ID×0.25 m (Thermo); and an Equity 5 30 m×0.25 μm ID×0.25 μm (SUPELCO).

Aliquots of 2 μl of extract were injected in splitless mode (30 sec) into an injector at 260° C.

The analysis program used was as follows: 30° C. for 4 min, then 5° C./min up to 280° C. and isotherm at 280° C. for 10 min.

Ionization of the molecules entering the mass spectrometer was obtained by electron impact at 70 eV, with a source temperature of 180° C.

Compounds of interest were identified by comparing the experimental ion spectra obtained with the NIST spectra database.

The analysis was focused on the molecules that varied during the animal's estrous cycle and more specifically during the pre-estrus and estrus stages.

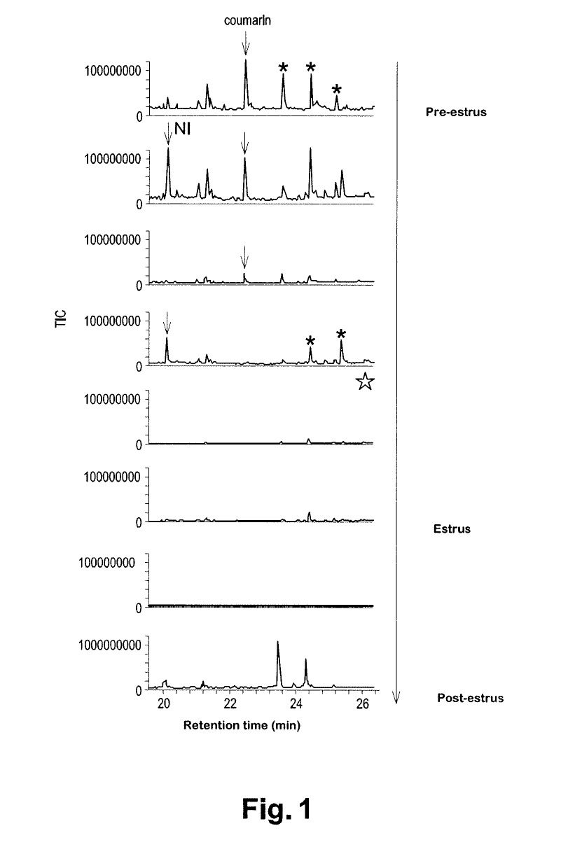

FIG. 1 illustrates how the molecules of interest were identified. The different stages of the cycle are shown on the right of the chromatograms by time scale. A star indicates the time of ovulation. Peaks with an asterisk above them correspond to compounds that are not specific to the stage, since they are found in other animals in the post-estrus phase; therefore these were not selected. Peaks with an arrow above them correspond to peaks specific to pre-estrus/estrus stages of a majority of animals, whether the compounds were identified (e.g. coumarin in FIG. 1) or not identified (e.g. the peak with “NI” above it in FIG. 1).

II—Results

### 1—Identification of the Molecules

About ten molecules were identified as potential estrus markers because of their presence in the pre-estrus and/or estrus stages in a majority of the cows.

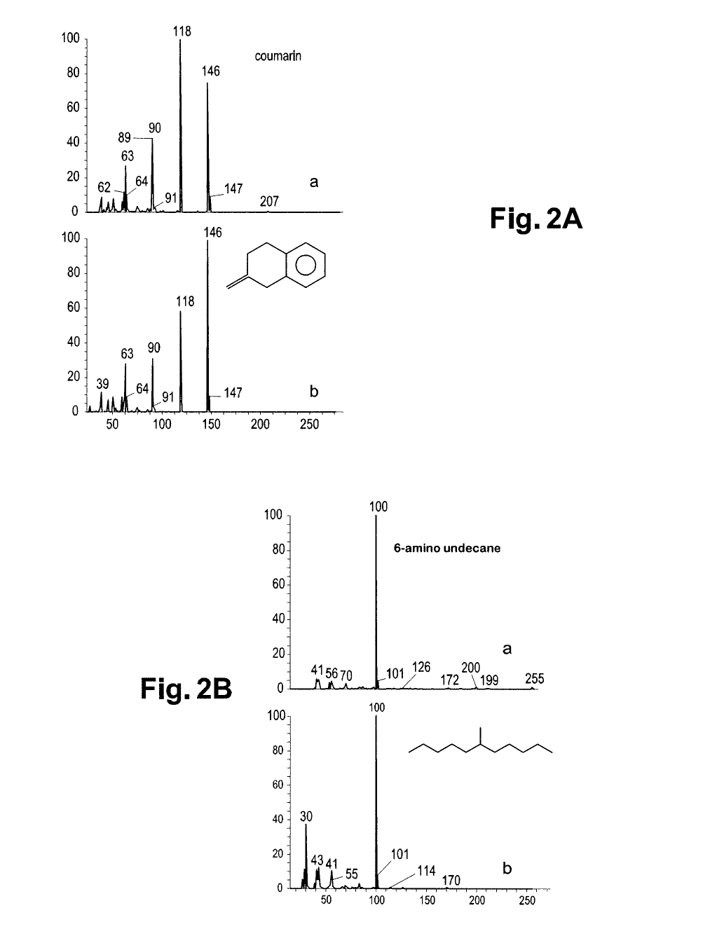

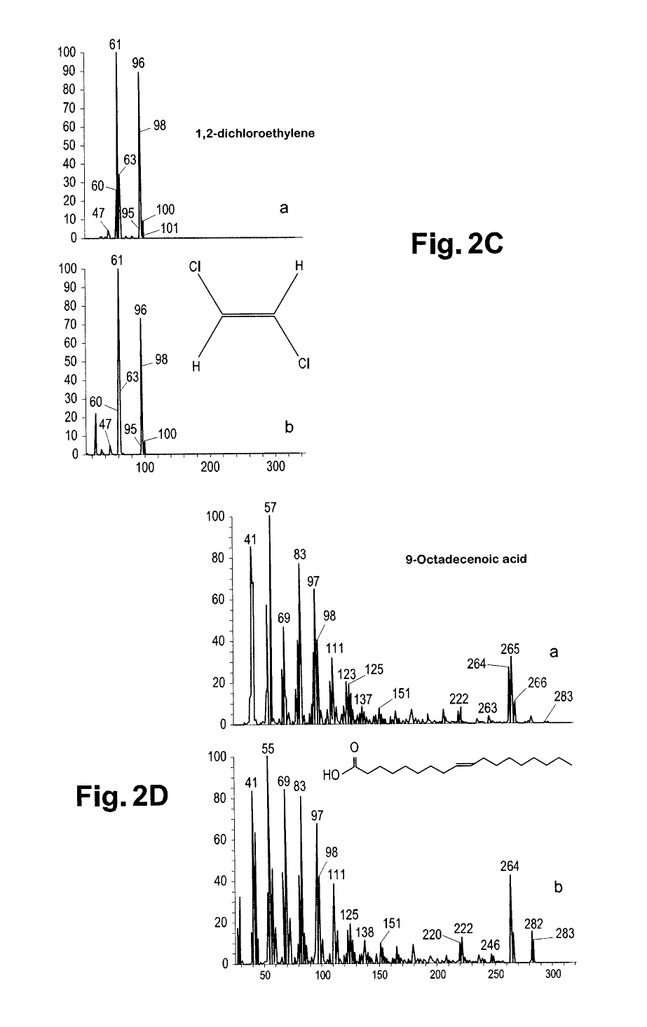

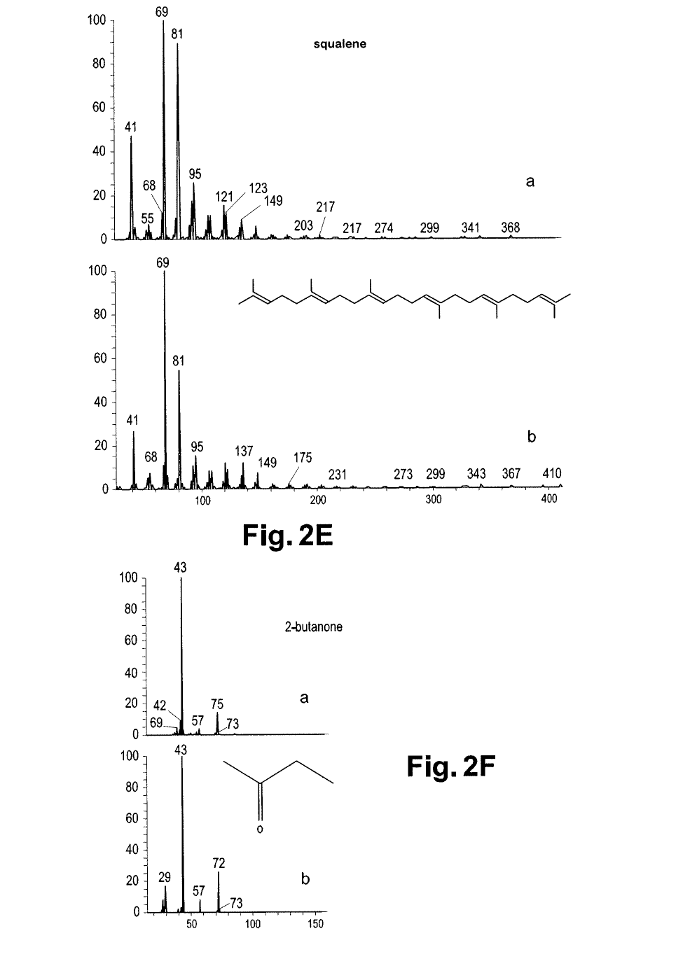

Six of these molecules—coumarin, 6-amino undecane, 1,2-dichloroethylene, squalene, 2-butanone and 9-octadecenoic acid—were identified by comparing their ion spectrum (experimental) with those of the NIST spectra database. FIGS. 2A, 2B, 2C, 2D, 2E and 2F compare the experimental ion spectra (a) of these molecules with the spectra of the NIST database (b).

Thus, in 5 of the 6 cows in pre-estrus and/or estrus stages coumarin was present in the cow urine of the second collection. In this collection it was identified in association with other molecules, not all of which have currently been identified. However, 6-amino undecane could be identified in pre-estrus in 5 of the six cows analyzed and in estrus in 2 of these. Coumarin was also found in estrus stage in one cow of the first collection.

1,2-dichloroethylene was identified in the analysis of the third collection made in the pre-estrus stage for half the cows studied and for three out of eight cows in estrus. Its presence was also detected in the urine of heifers during their first heats.

Squalene is the identified molecule that was found most regularly in the pre-estrus/estrus stages, regardless of the collection. Interestingly, this molecule was observed both in the urine in the stages defined above and in the vaginal mucus, biological fluids specific to estrus of cows.

9-octadecenoic acid is a fatty acid that was also found both in some urine (although less frequently than squalene) in the desired stages and in vaginal mucus.

2-butanone was also identified in an almost systematic way in the vaginal mucus of the cows collected.

2—Preparation of Compositions and Presentations

Three molecules, available on the market, were used to prepare the compositions used in the examples below, namely coumarin, squalene and 1,2-dichloroethylene.

To produce the compositions, the excipient used is a glycerol/water solution (50/50, v/v). This same excipient is used as control solution (Solution T).

2-1—Solution A

Solution A contains coumarin at a final concentration of 25 pg/ml. An initial dilution of the commercial coumarin was carried out in ethanol for a final concentration of 12.5 mg/ml, then two 100-fold dilutions were carried out in series in an ethanol solution to arrive at a mother solution A with a concentration of 12.5 ng/ml. One milliliter of this last dilution is diluted in 499 ml of excipient to obtain the final solution A.

2-2—Solution B

Solution B contains a mixture of squalene and 1,2-dichloroethylene at a total concentration of 25 pg/ml. In the same way as above, 12.5 mg of commercial compounds were taken up in 1 ml of ethanol to give a mother solution B at 12.5 mg/ml. These mother solutions were diluted 1000-fold in series 23 times in ethanol to arrive at a concentration of 12.5 ng/ml before final dilution in the excipient to obtain the final solution B.

2-3—Solution A+B

Solution A+B is obtained by diluting 1 ml of mother solutions A and B (at 12.5 ng/ml) in 498 ml of excipient.

2-4—Presentation

The solutions were presented in spray form, in sprayers used in cosmetology, made of aluminum lined with a neutral coating used in the agri-food industry to avoid having interfering substances released. Such a presentation makes it possible to present the composition to the bull while limiting losses in active molecules, i.e. the pheromones of interest, which are by nature very volatile. In effect, it can be deposited directly into the animal's nostrils and thus close to the target, namely the nervous system. Using a sprayer therefore makes it possible, by a noninvasive method, to limit the loss of active molecules, to control the dose delivered to the animal, and to be as close as possible to the target.

Alternatively, it is also possible to present the solution in cups that the animal will sniff and/or lick. It is also possible to use the solution to brush the genitals, amongst others, of the cow that must be served, or the teaser used in the context of collecting the bull's semen.

### EXAMPLE I

I—Materials and Methods

### 1—Animals

The first study involved a group of ten Prim Holstein or Normande bulls, 4 to 5.5 years old. These animals had been recruited in the “animal insemination” sector from the age of 2 to 3 months and had never been in contact with female cattle since then. They had always been kept in individual boxes (pre-testing period or production period) or in groups of 4 to 6 animals of the same age and sex (lay-off period). Food and accommodation conditions were always identical for the test animals, before and during the test.

Before the test the bulls were divided into 2 groups according to their libido, defined based on their behavior observed before the test in the covering barn. Group 1 corresponds to animals with “normal” libido, i.e. whose reaction and ejaculation times are consistent with those of all the stud bulls. Group 2, with “low libido”, consists of breeders considered “slow” or “very slow” until interest in the teaser is shown and an ejaculate is obtained.

### 2—Solution Used

In this example I, solution B was tested.

The solution containing the selected molecules (Solution B in Table 1) was presented in the form of an experimental spray in sprayers.

A control version (solution T in Table 1) was produced, using only the excipient also used for making up the solution of the active molecules in solution B.

The sprayers bore code numbers (1, 2) whose meaning was unknown to the bull herders, except for the person responsible for administering the treatment at the beginning of the collection.

### 3—General Organization of the Tests: Distribution of the Solutions

Table 1 below lists the experiments carried out for each group of bulls.

The tests lasted a total of 6 weeks. Only one person, always the same, was authorized to spray the solutions in the bulls' nostrils.

The first two weeks were devoted to training the bulls in the collection schedule, stabilizing sperm reserves and estimating the production potential of the individuals. They made it possible to ensure the pertinence of the distribution between the 2 groups: significant difference in libido estimated by the reaction and ejaculation times and no difference between production parameters.

The next four weeks formed the actual experimental phase, and were programmed using a latin square: each group was presented alternately with the control and the treatment and each bull also was its own control.

During the two weeks of adaptation the bulls only received the control spray containing the excipient, at a rate of one spray per nostril. Then, depending on their assignment in the experimental protocol, the animals received either the control spray or an experimental spray.

As the molecules used are very volatile, the collection always began with the “control” group in order not to saturate the atmosphere of the collection room with active molecules. All the animals received the treatment when they arrived in the covering barn, which was done randomly within each group.

TABLE 1
General organization of the tests for the adult bulls

| | Week 1 | Week 2 | Week 3 | Week 4 | Week 5 | Week 6 |
|---|---|---|---|---|---|---|
| Group 1 | Solution T | Solution T | Solution T | Solution B | Solution T | Solution B |
| Normal | Adaptation | Adaptation | Experiment | Experiment | Experiment | Experiment |
| libido | | | | | | |
| Group 2 | Solution T | Solution T | Solution B | Solution T | Solution B | Solution T |
| Low libido | Adaptation | Adaptation | Experiment | Experiment | Experiment | Experiment |

Solution T: control solution, containing only the solvent
Solution B: solution containing the pheromones according to the invention and the solvent

### 4—Recording Behavioral Parameters

Throughout the tests, the bulls of the tests were subjected to a weekly schedule of 3 ejaculate collections: Monday, Wednesday and Friday.

The preparation of the bulls was carried out systematically, following the schema described by Amann (see the definition above).

To evaluate the libido, it was therefore necessary before collecting each ejaculate, to record these three times, the reaction time and the time to ejaculation, being measured with a stopwatch.

The teaser remained the same during a single collection session. However, if the bull had shown no reaction after 10 minutes the teaser could be changed.

The team leader was the only person authorized to spray the treatments based on the animals' assignment to each of the groups and to measure the various times.

Libido parameters: The reaction (Treact) and ejaculation (Tejac) times described above were measured (in seconds) at each mounting for each bull. Manifestations of Flehmen, thrusting and the emissions of pre-sperm fluids were recorded.

Throughout the test, three persons measured the various times.

Similarly, the quality of the mounting was noted: none, weak, medium, strong, very strong.

For the other aspects, collections took place under the routine conditions in force in the stud.

### 5—Evaluating Biological Parameters

Once collected using an artificial vagina, the semen was sent directly to the laboratory to be processed under normal working conditions.

The quantitative—namely the volume (mL), concentration (billion spz/ml), and total number of spermatozoa per ejaculate (in billions)—and qualitative—namely the % of live spermatozoa and the quality of movement or motility—biological parameters were recorded for each ejaculate collected.

### 6—Statistical Analysis

A comparison of each group of bulls, i.e. Groups 1 and 2, was performed during the adaptation period to make sure that the only differences related to the animals' libido.

Then a first Proc GLM analysis of variance (with and without weeks of adaptation) on the overall population was performed using the SAS software and involved:
the libido parameters, i.e. the reaction time and the time to ejaculation,
the biological parameters, i.e. the volume, concentration, total number of spermatozoa, the percentage of living spermatozoa, the movement quality.

The following variation factors were taken into account in the model: Period, Week, Bull, Treatment, 2-by-2 Interactions.

A second intra-group Proc GLM analysis, without the weeks of adaptation, was performed to see if the treatment effects were similar according to the bulls' initial libido. Finally, the effects of the mixture on the males, taken individually, were analyzed.

II—Results

### 1—Initial Comparison of the Groups of Bulls (Before Treatment)

The performance of the 2 groups of bulls (G1: normal libido, G2: low libido) was compared based on all the parameters recorded during the adaptation period. Observation involved 6 collections, i.e. a total of 30 ejaculates per group. The results obtained are listed in Table 2 below.

TABLE 2
Comparison of the means and standard deviations observed
for the libido and production parameters between the 2 groups of bulls during
the adaptation period.

| | Treact | Tejac | Vol | C.° | No. spz | Mob |
|---|---|---|---|---|---|---|
| G1 | 89 ± 180 | 323 ± 280 | 6.7 ± 1.4 | 1.44 ± 0.3 | 9.4 ± 2.3 | 63 ± 3.1 |
| G2 | 320 ± 302 | 780 ± 478 | 7 ± 1.6 | 1.45 ± 0.32 | 10.3 ± 3.4 | 64 ± 3.1 |
| p | <0.0008 | <0.0001 | NS | NS | NS | 0.07 |

Vol: Volume in mL
C.°: concentration in billion spermatozoa/mL
No. spz: number of spermatozoa (in billions)
Mob: mobility
NS: Not significant

The observations of the two groups of bulls made it possible to note that they were significantly different with regard to the libido.

Thus, reaction and ejaculation times are on average 320 seconds and 780 seconds for group 2, compared to 89 seconds and 323 seconds for group 1, which is in line with expectations.

In contrast the production parameters, in particular the quantitative data, are comparable between the two groups.

### 2—Analyzing the Impact of the Treatments

2-1—On All the Bulls

The impact of the treatment was analyzed first of all for all the bulls, regardless of the group they were initially assigned to.

The analysis covered the 4-week period of experiments. The libido and production parameters of the 60 “control” ejaculates and the 60 “pheromone” ejaculates were compared. The results are presented in Table 3 below, which shows the libido and production parameters of the ejaculates collected, with or without treatment, for all the bulls.

TABLE 3
Comparison of the libido and production parameters of the
ejaculates collected during the experimental period.

| | N | Treact | Tejac | Vol | C.° | No. spz | Mob |
|---|---|---|---|---|---|---|---|
| Ctl | 60 | 310 ± 383 | 707 ± 637 | 7 ± 1.4 | 1.47 ± 0.2 | 10.2 ± 2.2 | 63. ± 4 |
| Ph+ | 60 | 232 ± 342 | 615 ± 518 | 7.2 ± 1.4 | 1.56 ± 0.22 | 11.1 ± 2.4 | 64 ± 4 |
| Δ | | −77 sec | −91 secs | +0.15 | +0.09 | +0.9 | |
| | | −25% | −13% | +2% | +6% | 9% | |
| p | | =0.08 | =0.06 | NS | =0.008 | 0.02 | |

N: number of ejaculates
Δ: difference between the 2 variables
p: probability out of 1 that the observed event is due to chance

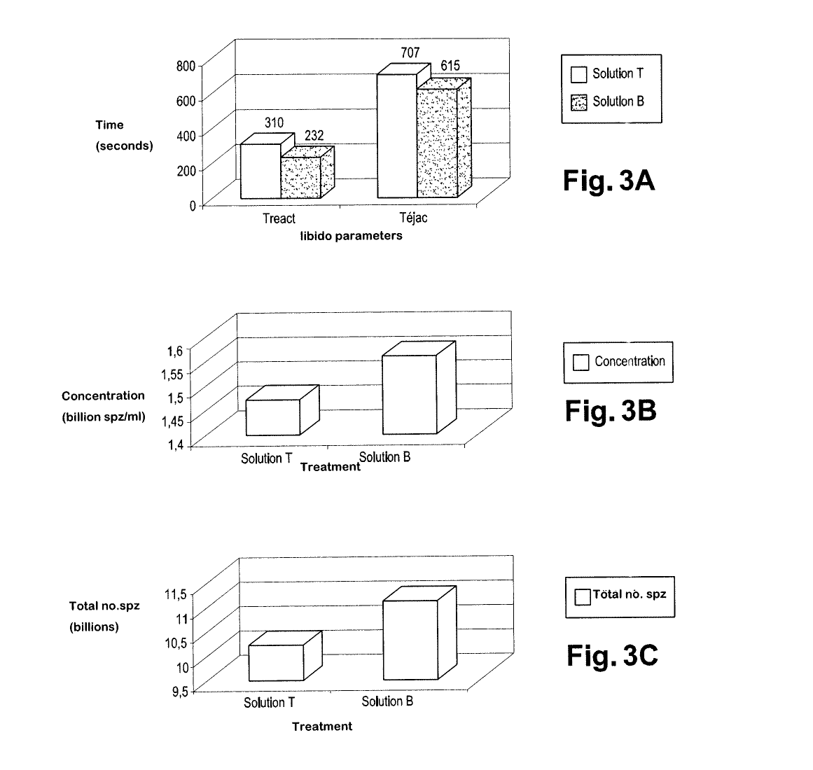

Taking the 10 bulls of the test as whole, the use of the molecules is reflected by a reduction in the reaction (Treact) and ejaculation (Tejac) times of 25% and 13% respectively (FIG. 3A).

In parallel, the concentration)(C.° and total number of spermatozoa collected per ejaculate (No.spz) improves by 6% (p=0.008) and 9% (p=0.02) respectively (FIGS. 3B and 3C), while the volume does not vary significantly (+2%).

The individual “bull” effect was significant for all the parameters analyzed. The ‘individual “bull” effect’ means the behavior specific to each animal and likely to influence the parameters measured.

No “bull*treatment” interaction was identified. All bulls exhibit a more or less marked improvement thanks to the treatment, said improvement being due solely to the treatment.

2-2—On the “Normal” Libido Bulls (G1)

To compare the effect of the molecules on bulls with normal libido, a statistical analysis (Proc GLM, SAS) was performed within the group during the experimental period.

The variation factors considered were the individual “bull” effect, the treatment and their 2-by-2 interaction.

The results are presented in Table 4 below.

TABLE 4
Comparison of the libido and production parameters of ejaculates
collected during the experimental period for the group 1 bulls.

| | N | Treact | Tejac | Vol | C.° | No. spz | Mob |
|---|---|---|---|---|---|---|---|
| Solution T | 30 | 45 ± 36 | 271 ± 140 | 7.07 ± 1.3 | 1.42 ± 0.21 | 10 ± 2.2 | 63 ± 3.6 |
| Solution B | 30 | 38 ± 32 | 255 ± 64 | 7.13 ± 1.1 | 1.56 ± 0.23 | 11.1 ± 2.3 | 64 ± 3.8 |
| p | | P< | NS | NS | P < 0.004 | P = 0.07 | |

It can be seen that the “bull” effect has a significant impact on the reaction time but has no impact on the ejaculation time. This is also noted for the concentration.

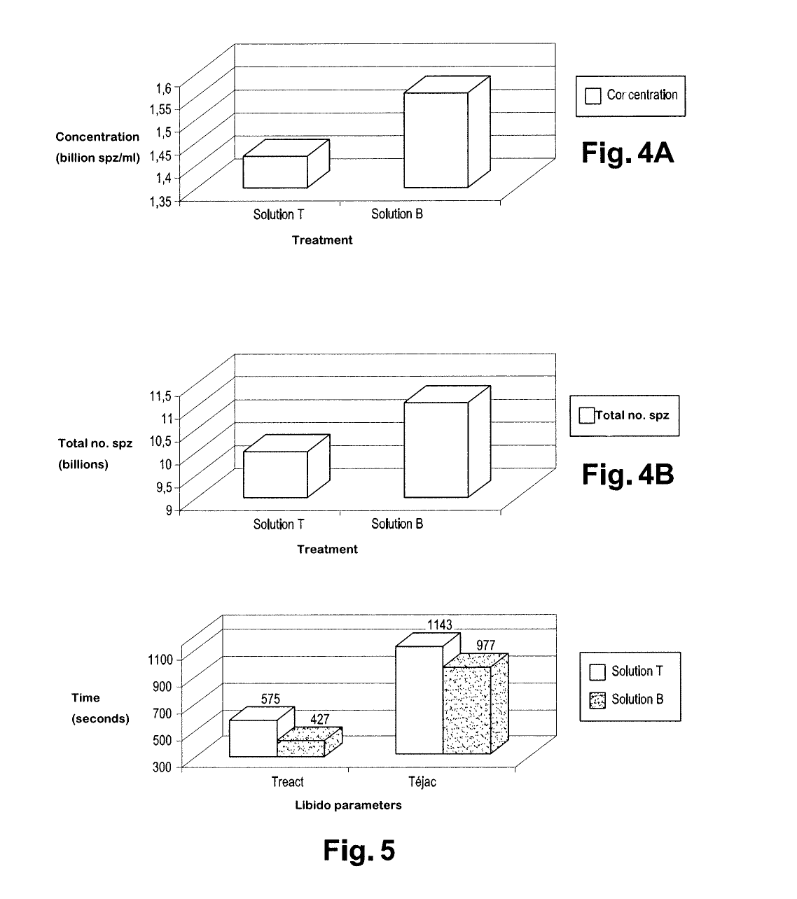

The use of pheromones reduces the reaction time by 15%, and allows an increase in the concentration of ejaculates (FIG. 4A) of 10% (p<0.004) and in the total number of spermatozoa (FIG. 4B) of 11% (p=0.07).

2-2—On the “Low” Libido Bulls (G2)

The model applied to the G1 group was then applied to the other group of bulls, G2. The overall results are presented in Table 5 below.

TABLE 5
Comparison of the libido and production parameters of ejaculates
collected during the experimental period for the group 2 bulls.

| | N | Treact | Tejac | Vol | C.° | No. spz | Mob |
|---|---|---|---|---|---|---|---|
| Solution T | 30 | 575 ± 389 | 1143 ± 642 | 7 ± 1.4 | 1.52 ± 0.27 | 10.4 ± 2.3 | 63.5 ± 4.3 |
| Solution B | 30 | 427 ± 398 | 977 ± 522 | 7.2 ± 1.6 | 1.55 ± 0.21 | 11.1 ± 2.5 | 64 ± 4.1 |
| Δ | | −148 sec | −165 sec | 0.23 | +0.03 | +0.67 | |
| | | −26% | −14% | +3% | +3% | +6% | |
| p | | P = 0.1 | P = 0.07 | NS | NS | NS | NS |

The individual “bull” effect appears significant for all the parameters.

The treatment only has an impact on the libido parameters (FIG. 5): 26% improvement for Treact (p=0.1) and 14% for Tejac (p=0.07).

The “burtreatment” interaction tends to be significant (p=0.09) for the concentration. This observation is due to the fact that the spermatozoa concentration of one bull out of the 5 in the group declined markedly due to the treatment (1.76 billion spz/ml as a control versus 1.55 billion/ml with pheromones) whereas for the other individuals the treatment induces an increase in this datum but in smaller proportions.

The other parameters do not seem to be influenced.

2-4—On the Individual “Bull” Effect

The “bull” effects were analyzed for each individual and each parameter as a function of the treatment and are presented in Tables 6 and 7 below.

TABLE 6
Effect of the treatment for each bull in group 1 on the libido
and production parameters

| Animal | Treatment | Treact | Tejac | Vol | C.° | No. spz | Mob |
|---|---|---|---|---|---|---|---|
| Vagan | Solution T | 53 | 349 | 7 | 1.6 | 11.2 | 64 |
| | Solution B | 59 | 323 | 6.3 | 1.8 (a) | 11.8 | 65 |
| Valier | Solution T | 59 | 262 | 7.6 | 1.4 | 10.7 | 63.3 |
| | Solution B | 71.2 | 262 | 7.4 | 1.5 | 11 | 64.2 |
| Valrus | Solution T | 25.3 | 239 | 6.9 | 1.4 | 9.8 | 63 |
| | Solution B | 20 | 255 | 7.5 | 1.5 | 11.2 | 65 |
| Vathi | Solution T | 58 | 281 | 6.2 | 1.5 | 9.2 | 62 |
| | Solution B | 21 (b) | 209 (c) | 7.3 d | 1.6 (e) | 12 (f) | 62 |
| Volcain | Solution T | 29 | 224 | 7.7 | 1.2 | 9.1 | 62.5 |
| | Solution B | 18 | 223 | 7.1 | 1.3 | 9.4 | 63 |

(a) p = 0.07;
(b) p = 0.06;
(c) p = 0.03;
d p = 0.005;
(e) p = 0.05;
(f) p = 0.003

In group 1, it is observed that the reaction times of 2 bulls out of the 5 increase as a result of the treatment. However, as a result of the treatment, the cumulative reaction time for the 5 animals is reduced relative to the control (189.2 seconds with treatment against 224.3 seconds with no treatment, a time reduction of 15.6%).

The ejaculation time is improved for 2 animals, significantly for one (p=0.03), does not change for 2 others and tends to increase for the last. Cumulatively, the treatment enables the ejaculation time of the 5 bulls taken together to be reduced by 6.1% (1356 seconds without treatment against 1272 seconds with treatment).

TABLE 7
Effect of the treatment for each bull in group 2 on the libido and
production parameters

| Animal | Treatment | Treact | Tejac | Vol | C. ° | No.spz | Mob |
|---|---|---|---|---|---|---|---|
| vador | Solution T | 581 | 1144 | 8 | 1.6 | 12.4 | 65 |
| | Solution B | 564 | 1032 | 7.6 | 1.7 | 13.7 | 67 |
| vanzi | Solution T | 316 | 709 | 6 | 1.4 | 9.6 | 62.5 |
| | Solution B | 321 | 817 | 7.2 | 1.5 | 11 | 62.5 |
| Virtel | Solution T | 1099 | 2237 | 5 | 1.7 | 8.8 | 61 |
| | Solution B | 633 | 1630 | 5.1 | 1.6 | 7.7 | 60 |
| Voladi | Solution T | 472 | 910 | 7 | 1.7 | 11.7 | 65 |
| | Solution B | 392 | 834 | 7.2 | 1.7 | 12 | 67 |
| Vosac | Solution T | 405 | 713 | 8.4 | 1.14 | 9.6 | 64 |
| | Solution B | 225 | 571 | 8.5 | 1.29 | 11 | 63 |

In group 2, the treatment enables a cumulative improvement of 25.7% in the reaction time, and of 14.5% in the ejaculation time, i.e. 738 and 829 seconds respectively for the 5 bulls.

This means that the treatment enables 13 minutes 48 seconds to be saved out of the total time spent in the covering barn when the 5 bulls are part of the same mounting session.

### EXAMPLE II

I—Materials and Methods

### 1—Animals

The second study involved twenty young Montbeliard bulls, 15 to 18 months old, that were part of a group of animals tested for the sexual function (CFS). These bulls had been recruited from farms at the age of 2 to 3 months, and then raised under identical conditions with no contact with females.

In the absence of prior libido data, they were randomly divided into two groups, J and K, of 10 individuals.

### 2—Solution Used

Solution A+B was used and presented in the form of experimental spray in sprayers.

A control version (solution T in Table 8) was produced, using only the solvent also used for making up the solution of the active molecules in solution A+B.

The sprayers bore code numbers (1, 2) whose meaning was unknown to the bull herders, except for the person responsible for administering the treatment at the beginning of the collection.

### 3—General Organization of the Tests: Distribution of the Solutions

Table 8 below lists the experiments carried out for each group of bulls.

The test lasted 5 weeks, including one week of adaptation during which all the animals received the control solution and only one collection was carried out. The solution was presented in spray form, as in Example I above.

The solutions were systematically administered at the beginning of the collection to all the bulls in the form of two sprays of the mixture per nostril. As the molecules used are very volatile, the collection always began with the “control” group in order not to saturate the atmosphere of the collection room with active molecules. All the animals received the treatment when they arrived in the covering barn, which was done randomly within each group.

TABLE 8
General organization of the tests for the adult bulls

| | Week 1 | Week 2 | Week 3 | Week 4 | Week 5 |
|---|---|---|---|---|---|
| Group J | Solution | Solution | Solution | Solution | Solution |
| | T | T | A + B | T | A + B |
| | Adapta- | Experi- | Experi- | Experi- | Experi- |
| | tion | ment | ment | ment | ment |
| Group K | Solution | Solution | Solution | Solution | Solution |
| | T | A + B | T | A + B | T |
| | Adapta- | Experi- | Experi- | Experi- | Experi- |
| | tion | ment | ment | ment | ment |

### 4—Recording Behavioral and Production Parameters

Throughout the experimental period of the test, i.e. 4 weeks, all animals were subjected to a schedule of 2 weekly collections of one ejaculate (2*1).

The preparation of the bulls and the teaser, and the methods of administering the solution and collecting the ejaculates were the same as in Example I.

The observations and recording of libido and production parameters were the same as in Example I.

II—Results

### 1—Analyzing the Effects of the Treatment, the Group, the Bull Intra-Group and Their Interactions

The mean values obtained for each parameter measured for all 20 bulls taken together and as a function of the treatment are presented in Table 9 below. They relate to the 4 weeks of the experimental period.

TABLE 9
mean values of the parameters measured, as a function of the
treatment, for all the bulls

| Treatment | number | Treact | Tejac | Vol | C. ° | Nospz | Mob |
|---|---|---|---|---|---|---|---|
| Solution T | 80 | 33.5 | 347 | 3.36 | 1.01 | 3.39 | 56.34 |
| Solution | 80 | 31.1 | 297 | 3.23 | 1.09 | 3.45 | 56.02 |
| A + B | | | | | | | |
| p | | NS | P < 0.08 | NS | P = | NS | NS |
| | | | | | 0.11 | | |

With regard to the entire group of 20 bulls in the test, it appears that using the cocktail of pheromones tends to reduce the ejaculation time (347 seconds for the controls against 297 seconds for those treated, i.e. a time reduction of 14%) and to increase the mean concentration of the ejaculate (1.01 billion/ml for the control group against 1.09 billion/ml for the treated group, i.e. an increase of 9% (p<0.11)).

The other parameters do not seem to be changed by the treatment.

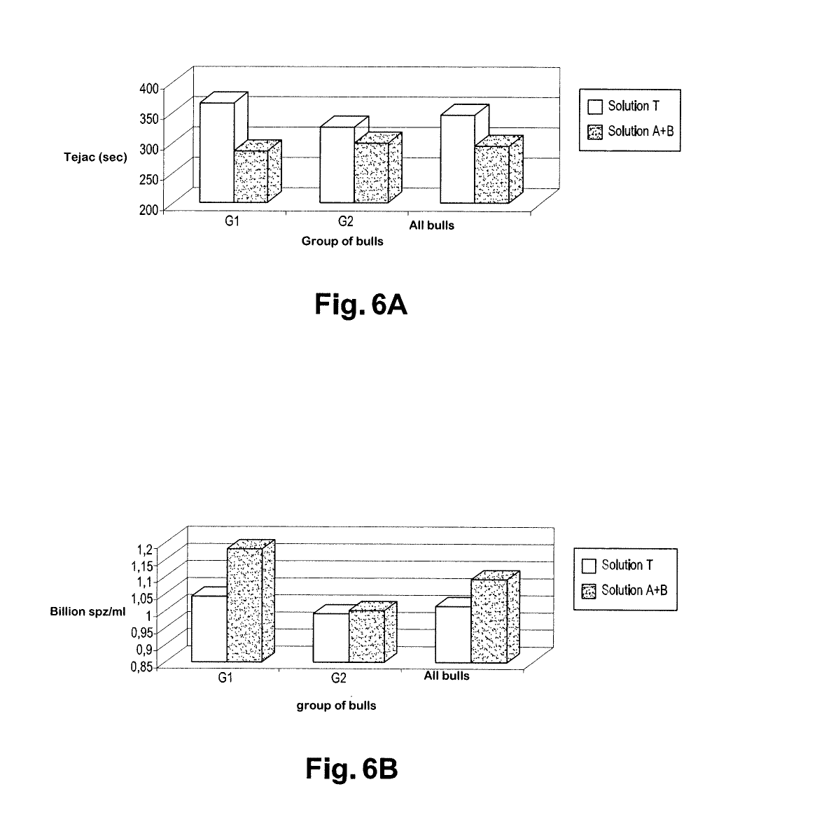

The impact of the treatment on the ejaculation time and concentration, for each group of bulls and overall, is shown in FIGS. 6A and 6B.

Thus, it is observed that, for all parameters measured, the individual “bull” effect appears very significant (p<0.0001). In contrast, no “bull*treatment” interaction is observed.

It appears that the effectiveness of the treatment is higher when the bulls' initial libido is low and the control ejaculation time is high.

Of the 20 bulls in the test, only one (T15) has atypical behavior and an ejaculation time that increases with the treatment.

Similarly, for the individuals with a control ejaculation time of less than 200 seconds, corresponding to a good initial libido, the treatment does not appear to significantly improve the libido.

### EXAMPLE III

I—Materials and Methods

### 1—Animals

The first study involved a group of ten Prim Holstein or Normande adult bulls, 4 to 5.5 years old. These animals had been recruited in the “animal insemination” sector from the age of 2 to 3 months and had never been in contact with female cattle since then. They had always been kept in individual boxes (pre-testing period or production period) or in groups of 4 to 6 animals of the same age and sex (lay-off period). Food and accommodation conditions were always identical for the test animals, before and during the test.

As in Example I, the bulls were divided into two groups according to their libido: Group N, “normal libido”, and Group L, “low libido”.

### 2—Solutions Used

Solution A was used and presented in the form of the experimental spray in sprayers.

A control version (solution T in Table 10) was produced, using only the excipient also used for making up the solution of the active molecules in solution A.

The sprayers bore code numbers (1, 2) whose meaning was unknown to the bull herders, except for the person responsible for administering the treatment at the beginning of the collection.

### 3—General Organization of the Tests: Distribution of the Solutions

Table 10 below lists the experiments carried out for each group of bulls.

The test lasted a total of 6 weeks.

The weeks were organized as in Example I.

TABLE 10
General organization of the tests for the adult bulls

| | Week 1 | Week 2 | Week 3 | Week 4 | Week 5 | Week 6 |
|---|---|---|---|---|---|---|
| Group N | Solution T | Solution T | Solution T | Solution A | Solution T | Solution A |
| Normal | Adaptation | Adaptation | Experiment | Experiment | Experiment | Experiment |
| libido | | | | | | |
| Group L | Solution T | Solution T | Solution A | Solution T | Solution A | Solution T |
| Low libido | Adaptation | Adaptation | Experiment | Experiment | Experiment | Experiment |

The solution was presented in spray form, as in Example I above.

The solutions were systematically administered at the beginning of the collection to all the bulls in the form of two sprays of the mixture per nostril.

As the molecules used are very volatile, the collection always began with the “control” group in order not to saturate the atmosphere of the collection room with active molecules. All the animals received the treatment when they arrived in the covering barn, which was done randomly within each group.

### 4—Recording Behavioral Parameters

The collection schedules, preparation of the bulls and recording of the behavioral parameters were carried out under identical conditions to those in Example I.

### 5—Evaluating Biological Parameters

The semen collection was carried out under identical conditions to those described in Example I.

The quantitative and qualitative biological parameters were recorded as in Example I.

II—Results

### 1—Initial Comparison of the Bulls

### 1-1 Comparison Between the Groups of Bulls

The performance of the two groups of bulls (Group N and Group L) was compared based on all the parameters recorded during the adaptation period.

Observation involved 5 collections of 2 ejaculates (rank 1 and rank 2), i.e. 42 ejaculates for Group N and 47 ejaculates for Group L, due to mating refusals and missing data.

The results are presented in Table 11 below and correspond to all the ejaculates collected, regardless of their collection rank:

TABLE 11
Comparison of the means and standard deviations observed for the libido and
production parameters between the 2 groups of bulls during the adaptation period.

| | Treact | Tejac | Vol | C. ° | No.spz | Mob |
|---|---|---|---|---|---|---|
| Group N | 139 ± 357 | 378 ± 361 | 5.2 ± 1.6 | 1.21 ± 0.48 | 6.3 ± 3.4 | 84.5 ± 3.3 |
| Group L | 895 ± 1041 | 1368 ± 1176 | 6.4 ± 3 | 1.37 ± 0.5 | 8.9 ± 5.3 | 84.5 ± 3.6 |
| p | <0.0001 | <0.0001 | 0.03 | NS | 0.012 | NS |

It is observed that the distribution of the bulls in the 2 groups is consistent because the animals in group L take significantly longer to excite (Treact=895 seconds for group L, against only 139 seconds for group N) and give sperm (Tejac=1368 seconds for group L, and 378 seconds for group N).

It is also observed that the quantitative parameters of the spermogram are significantly higher for the bulls in group L (p=0.03 for the volume, p=0.012 for the number of total spermatozoa and a concentration of 1.37 billion/ml for group L, against 1.21 billion/ml for group N).

Because of these differences between groups, the rest of the statistical analysis was done separately for the 2 groups of bulls, except for the qualitative parameters that do not differ.

### 2-2 Comparison Between the Bulls' Ejaculate Ranks

The analyses were then performed integrating the “ejaculate rank” factor.

2-2-1—Comparison Between Rank 1 Ejaculates for the Bulls of the Two Groups

The results obtained for each group of bulls are presented in Table 12 below.

TABLE 12
comparison of the libido and production parameters between the 2
groups of bulls for rank 1 ejaculates

| | Treact | Tejac | Vol | C. ° | Nospz | Mob |
|---|---|---|---|---|---|---|
| Group L | 1248 | 1724 | 7.1 | 1.65 | 11.65 | 85 |
| N = 22 | | | | | | |
| Group N | 68.3 | 339.4 | 5.25 | 1.37 | 7.24 | 85 |
| N = 23 | | | | | | |
| p | 0.0001 | 0.0001 | 0.02 | 0.05 | 0.004 | NS |

The differences observed between the 2 groups for all the ejaculates collected are more pronounced for the rank 1 ejaculates.

The libido parameters differ very significantly since the mean reaction time ranges from 1 minute 8 seconds for Group N to 20 minutes 48 seconds for the ‘slow’ animals, and the mean ejaculation time ranges from 5 minutes 39 seconds for the “normal” libido bulls to an average of 28 minutes 44 seconds for the Group L bulls.

The quantitative parameters of the spermogram also differ significantly between the groups, in favor of group L, whereas the qualitative parameters (% mobile) are identical.

2-2-2—Comparison Between Rank 2 Ejaculates for the Bulls of the Two Groups

The results obtained for each group of bulls are presented in Table 13 below.

TABLE 13
comparison of the libido and production parameters between the 2
groups of bulls for rank 2 ejaculates

| | Treact | Tejac | Vol | C. ° | Nospz | Mob |
|---|---|---|---|---|---|---|
| Group L | 508 | 976 | 5.64 | 1.06 | 5.92 | 82.2 |
| N = 22 | | | | | | |
| Group N | 207 | 415 | 5.24 | 1.05 | 5.47 | 82.3 |
| N = 23 | | | | | | |
| p | 0.1 | 0.02 | NS | NS | NS | NS |

For the rank 2 ejaculates, the differences between groups disappear and only the libido parameters tend to be significantly different (Treact, p<0.1 and Tejac, p<0.02). Thus, the mean ejaculation time of 6 minutes 55 seconds in Group N increases to 16 minutes 16 seconds in Group L. It is interesting to observe that in Group L the libido parameters improve during the 2^{nd} mounting whereas they deteriorate in Group N.

The quantitative and qualitative parameters of the spermogram are very similar between the 2 groups with regard to the 2^{nd} ejaculate.

### 3—Impact of the Treatments

### 3-1—Effects of the Treatment According to Group on Rank 1 Ejaculates

3-1-1—Group N (Bulls With Normal Libido)

In this group the treatment has a positive effect on the production parameters, whereas the libido parameters do not change significantly.

The “bull” effect is only apparent for the quantitative characteristics of the spermogram and no “bull*treatment” interaction (Bull*treat in Table 14) is shown.

The results are presented in Table 14.

TABLE 14
Means for the libido and production parameters and analysis of the
variance factors during the experimental period. Group N. (n = 30
observations per treatment)

| | Treact | Tejac | Vol | C. ° | Nospz | Mob |
|---|---|---|---|---|---|---|
| Bull | NS | NS | P < | P < 0.0001 | P < 0.0001 | NS |
| | | | 0.0001 | | | |
| Treatment | NS | NS | NS | P < 0.05 | P < 0.05 | P < |
| | | | | | | 0.02 |
| Bull * | NS | NS | NS | NS | NS | NS |
| treat | | | | | | |
| Solution T | 84.7 | 316 | 5.03 | 1.35 | 6.73 | 84.5 |
| Solution A | 94.4 | 340 | 5.15 | 1.50 | 7.75 | 86.3 |
| Δ(A − T) | +9.7 s | +24 s | 0.12 | 0.15 | +1.02 | +1.8 |

3-1-2—Group L (Bulls With Low Libido)

In this group (n=30 observations per treatment), the individual “bull” effects are clearly apparent, whatever the parameter analyzed, while the treatment is significant for the percentage of mobile spermatozoa, the ejaculate volume and the total number of spermatozoa collected (p<0.07).

Thus, the treatment reduces the ejaculation time per bull by 393 seconds, i.e. 6 minutes 33 seconds. These data are summarized in Table 15.

TABLE 15
Means for the libido and production parameters and analysis
of the variance factors during the experimental period.

| | Treact | Tejac | Vol | C. ° | Nospz | Mob |
|---|---|---|---|---|---|---|
| Bull | P < 0.0001 | P < 0.0001 | P < 0.0001 | P < 0.0001 | P < 0.0001 | P < 0.07 |
| Treatment | NS | NS | P < 0.06 | NS | P < 0.07 | P < 0.05 |
| Bull * treat | NS | P < 0.07 | NS | NS | NS | NS |
| Solution T | 1078 | 2179 | 6.6 | 1.51 | 9.89 | 86.2 |
| Solution A | 846 | 1786 | 5.88 | 1.47 | 8.18 | 84.3 |
| Δ(A − T) | −232 s | −393 s | −0.72 | −0.04 | −1.71 | −1.9 |

### 3-2—Effects of the Treatment According to Group on Rank 2 Ejaculates

3-2-1—Group N (Bulls With Normal Libido)

In the “normal” libido bulls (n=30 observations per treatment), the molecules used tend to reduce the ejaculation time (p<0.07) and also the concentration and number of spermatozoa (p<0.01).

They have a positive effect (p<0.02) on the percentage of motile spermatozoa.

The impact of the bull is only apparent in the quantitative parameters of the spermogram, whereas there is no apparent “bull*treatment” interaction (Table 16).

TABLE 16
Means for the libido and production parameters and analysis of the
variance factors during the experimental period, 2^{nd} ejaculate.

| | Treact | Tejac | Vol | C. ° | Nospz | Mob |
|---|---|---|---|---|---|---|
| Bull | NS | NS | P < 0.0001 | P < 0.0006 | P < 0.0001 | NS |
| Treatment | NS | P < 0.07 | NS | P < 0.01 | P < 0.01 | P < 0.02 |
| Bull * treat | NS | NS | NS | NS | NS | NS |
| Solution T | 180 | 415 | 4.65 | 1.22 | 5.76 | 82.2 |
| Solution A | 71 | 258 | 4.31 | 0.99 | 4.25 | 84.5 |
| Δ(A − T) | −109 s | −157 s | −0.34 | −0.23 | −1.51 | +2.3 |

3-2-2—Group L (Bulls With Low Libido)

In this batch of animals (n=30 observations per treatment), only the individual “bull” effect is apparent for all the parameters studied, except for the percentage of motile spermatozoa, which is the only one on which the treatment has an impact. Paradoxically, the treatment tends to increase the libido parameters but not significantly. These data are summarized in Table 17.

TABLE 17
Means for the libido and production parameters and analysis of the
variance factors during the experimental period, 2^{nd} ejaculate.

| | Treact | Tejac | Vol | C. ° | Nospz | Mob |
|---|---|---|---|---|---|---|
| Bull | P < 0.01 | P < 0.0001 | P < 0.0001 | P < 0.01 | P < 0.0001 | NS |
| Treatment | NS | NS | NS | NS | NS | P < 0.01 |
| Bull * treat | NS | NS | NS | NS | NS | NS |
| Solution T | 312 | 868 | 5.74 | 0.91 | 5.07 | 84.6 |
| Solution A | 494 | 1137 | 5.28 | 0.88 | 4.84 | 81.9 |
| Δ(A − T) | +182 | +269 | −0.46 | −0.03 | −0.23 | −2.7 |

### EXAMPLE IV

I—Materials and Methods

### 1—Animals

Groups of ten adult bulls of different breeds were tested on several test sites. Each group was divided into two categories: control animals and treated animals. At each site, the bulls were assigned evenly between the two groups taking age and race data into account, so that only libido differences were discriminant.

As in Example III, the bulls were divided according to their libido: Group N, “normal libido”, and Group L, “low libido”.

For each site, the races treated and the collection schedules are shown in Table 18.

TABLE 18
Races and collection schedules at each site

| Site | Number | Races | Collection schedules |
|---|---|---|---|
| 1 | 5 controls | Holstein/Normande | 3*1 |
| | 5 treated | | |
| 2 | 5 controls | Holstein/Normande | 3*2 |
| | 5 treated | | |
| 3 | 5 controls | Limousine | 2*1 |
| | 5 treated | | |
| 4 | 5 controls | Holstein // Bl d'Aquitaine | 2*1 |
| | 5 treated | Simmental | |
| 5 | 5 controls | Holstein | 2*1 |
| | 5 treated | | |

where x*y expresses a number x of weekly collections of y successive ejaculates.

### 2—Solutions Used

A control solution (solution T in Table 19), comprising glycerol and water in respective proportions of 50/50 by volume, was used.

The other solutions, comprising the molecule or molecules according to the invention, were diluted in this control solution.

Different concentrations of the active molecule or mixture of active molecules were tested.

The compositions tested are described in Table 19.

TABLE 19

| Site | Composition | Code |
|---|---|---|
| All sites | Control solution (glycerol:water, 50:50) | T |
| 1 | 1,2-dichloroethylene 2.5 pg/mL + | C1 |
| | squalene 2.5 pg/mL | |
| | in the control solution | |
| 2 | 1,2-dichloroethylene 250 pg/mL + | C2 |
| | squalene 250 pg/mL | |
| | in the control solution | |
| 3 | 1,2-dichloroethylene 25 pg/mL + | C3 |
| | squalene 25 pg/mL + | |
| | 2-butanone 25 pg/mL | |
| | in the control solution | |
| 4 | 9-octadecenoic acid 25 pg/mL | C4 |
| | in the control solution | |
| 5 | 2-butanone 25 pg/mL | C5 |
| | in the control solution | |

All these compositions were presented in spray form, in sprayers made of aluminum lined with a neutral coating to avoid having interfering substances released.

On each site the stud team had two coded sprayers. One contained only the control solution, and the other had the composition containing one or a mixture of molecules according to the invention. Only the team leader could differentiate between the two sprayers, but did not know their content.

### 3—General Organization of the Tests: Distribution of the Solutions

Table 20 below lists the experiments carried out for each group of bulls, N and L.

The test lasted a total of 6 weeks. The first two weeks were devoted to training the bulls in the collection schedule, stabilizing sperm reserves and estimating the production potential of the individuals. They made it possible to ensure the pertinence of the distribution between the two groups: significant difference in libido estimated by the reaction and ejaculation times and no difference between production parameters. The next 4 weeks formed the actual experimental phase, and were programmed using a latin square: each group was presented alternately with the control spray and the treatment and each bull also was its own control.

TABLE 20
General organization of the tests per week

| | Adaptation | | Experiment | | | |
|---|---|---|---|---|---|---|
| | Wk. 1 | Wk. 2 | Wk. 3 | Wk. 4 | Wk. 5 | Wk. 6 |
| Group | Solution T | Solution T | Solution T | Comp. | Solution | Comp. |
| N | | | | Cx | T | Cx |
| Group | Solution T | Solution T | Comp. Cx | Solution | Comp. | Solution |
| L | | | | T | Cx | T |

where
Comp. Cx denotes one of the compositions C1 to C5, depending on the test site

In order not to saturate the atmosphere of the covering barn with active molecules and to avoid distorting the results, the control solution was systematically used before the compositions Cx.

### 4—Recording Behavioral Parameters

The collection schedule was realized as indicated in Table 18 above. The recording of behavioral parameters was carried out under similar conditions to those of Example I.

For site 2, the parameters relating to the first and second ejaculate were taken into account.

### 5—Statistical Analysis

A statistical analysis was performed according to the method described for Example I above, for the libido parameters.

II—Results

### 1—Effects of the Treatment on the Libido Parameters

### 1-1—Effects on the Reaction Time (Treact)

The effects of the treatment for each site and on all 10 bulls tested are presented in Table 21.

TABLE 21
Effects of each composition tested on the reaction time (in seconds)

| | Controls | | | | Treated | | | | | |
|---|---|---|---|---|---|---|---|---|---|---|
| | | Std. | | | | Std. | | | Mean diff. | |
| Comp. | Mean | dev. | Max. | Min. | Mean | dev. | Max. | Min. | (%) | p |
| C1 | 261.5 | 533 | 2696 | 6 | 166 | 273.4 | 1360 | 6 | −36 | 0.09 |
| C2 | 251 | 842.4 | 5700 | 2 | 460 | 1240.4 | 6600 | 2 | +83 | |
| Ejac. 1 | | | | | | | | | | |
| C2 | 76.3 | 125.2 | 720 | 2 | 72.2 | 109.2 | 445 | 2 | −5.4 | NS |
| Ejac. 2 | | | | | | | | | | |
| C3 | 315 | 390.5 | 1498 | 10 | 231 | 334.7 | 1550 | 2 | −27 | 0.13 |
| C4 | 16.7 | 14.7 | 48 | 1 | 11.5 | 11.5 | 45 | 1 | −31 | 0.03 |
| C5 | 807 | 912.8 | 3600 | 4 | 549 | 601 | 2100 | 6 | −33 | 0.002 |

where
Comp. means Composition,
Ejac. means Ejaculate,
Mean means Mean,
Mean diff. means Mean difference,
NS means Not Significant

For all the compositions tested, except for composition C2, it is observed that the treatment enables a noticeable reduction in the reaction time (27% to 36% reduction on average compared to the controls).

These results show that the C1, C3, C4 and C5 compositions according to the invention do not change the minimum reaction times of the bulls. In contrast, it can be seen that they induce a clear reduction in the maximum reaction times. In addition, they tend to reduce the variability between ejaculates and homogenize the reaction times observed.

These results thus demonstrate that even at a concentration as low as 2.5 pg/ml (composition C1), the molecules according to the invention are able to effectively stimulate the libido of bulls.

For composition C2, in which the molecules are present at a concentration of 250 pg/ml, an improvement in the reaction time for rank 2 ejaculate is still observed.

### 1-2—Effects on the Ejaculation Time (Tejac)

The effects of the treatment for each site and on all 10 bulls tested are presented in Table 22 below.

TABLE 22
Effects of each composition tested on the ejaculation time (in seconds)

| | Controls | | | | Treated | | | | | |
|---|---|---|---|---|---|---|---|---|---|---|
| | | Std. | | | | Std. | | | Mean diff. | |
| Comp. | Mean | dev. | Max. | Min. | Mean | dev. | Max. | Min. | (%) | p |
| C1 | 678.5 | 772.5 | 3444 | 184 | 535 | 441 | 2385 | 164 | −21 | 0.03 |
| C2 | 474.5 | 882.7 | 5820 | 43 | 736 | 1283 | 6660 | 100 | +55 | |
| Ejac. 1 | | | | | | | | | | |
| C2 | 427 | 501.4 | 3040 | 30 | 308.6 | 217 | 980 | 48 | −28 | |
| Ejac. 2 | | | | | | | | | | |
| C3 | 1138 | 388 | 2344 | 520 | 985 | 411.5 | 2160 | 562 | −13 | 0.02 |
| C4 | 35.6 | 21.1 | 83 | 5 | 29.2 | 18.9 | 78 | 5 | −18 | 0.06 |
| C5 | 1232.5 | 1308.8 | 3600 | 135 | 935 | 726 | 2877 | 165 | −24 | 0.005 |

where
Comp. means Composition,
Ejac. means Ejaculate,
Mean means Mean,
Mean diff. means Mean difference

The above results show that, except for composition C2, the tested compositions according to the invention significantly reduce ejaculation time by 13 to 28% depending on the composition. This is also true for composition C1, in which the concentration of molecules is as low as 2.5 pg/ml.

This effect is also observed significantly in the rank 2 ejaculate for composition C2.

As for the reaction time, it is observed that the compositions according to the invention mainly induce a decrease in the maximum ejaculation times, as well as in the variability between the bulls tested.

These results support the conclusion that the molecules tested, at the concentrations tested, have a significant biological effect on the libido of bulls.

At a total concentration of the molecules specific to the estrus as high as 500 pg/mL (composition C2), the beneficial effect on reaction and ejaculation times is also observed in the rank 2 ejaculate collected on the same day as the rank 1 ejaculate. This beneficial effect is delayed in time with respect to the administration of the composition, but is nevertheless significant.

### 2—Analysis of the Individual “Bull” Effects and “Bull-Treatment” Interactions

Whatever the sites and parameters considered, it is apparent from the results obtained that the individual “bull” effect has always appeared very significant. No “bull/treatment” interaction has emerged as significant, including for the libido parameters, reaction time and ejaculation time, when the positive effect of the treatment was noted. These data were consistent regardless of the molecules or their mixtures used, which supports the idea that the molecules according to the invention act identically on almost all bulls, although in varying proportions depending on the initial libido of the animals.

To illustrate these data, the “bull” effects observed at site 3 on the libido parameters influenced by the treatment are presented below as an example, as they are representative of what is obtained on all the other sites.

Table 23 below shows the results obtained in terms of reaction time for each bull, depending on the presence or absence of the treatment. Each mean is calculated from 4 ejaculates (schedule 2*1) collected during the 4 weeks of the experimental period.

TABLE 23
Illustration of the individual “bull” effect and the absence of a
“bull/treatment” interaction on the reaction time (Treact), in seconds, on site 3
(composition C3)

| | Controls | | | | Treated | | | | | |
|---|---|---|---|---|---|---|---|---|---|---|
| | | Std. | | | | Std. | | | Mean | Mean |
| Treact | Mean | dev. | Min. | Max. | Mean | dev. | Min. | Max. | diff. | diff. % |
| bull 1 | 43 | 24 | 20 | 65 | 38.3 | 21.08 | 16 | 60 | −4.7 | −11 |
| bull 2 | 55 | 31 | 27 | 89 | 44 | 32.6 | 14 | 85 | −11 | −20 |
| bull 3 | 79 | 21.7 | 57 | 102 | 59.8 | 12.9 | 48 | 77 | −19.2 | −24 |
| bull 4 | 539 | 187 | 374 | 733 | 319 | 244.8 | 82 | 638 | −220 | −41 |
| bull 5 | 24.3 | 13.7 | 10 | 42 | 5 | 2.4 | 2 | 8 | −19.3 | −79 |
| bull 6 | 770 | 584 | 200 | 1463 | 884 | 452 | 598 | 1550 | 114 | 15 |
| bull 7 | 897 | 501 | 379 | 1498 | 530 | 436 | 145 | 1124 | −367 | −41 |
| bull 8 | 139 | 48.7 | 74 | 182 | 93 | 50 | 63 | 167 | −46 | −33 |
| bull 9 | 115.3 | 104 | 47 | 270 | 56 | 77 | 7 | 170 | −59.3 | −51 |
| bull 10 | 487 | 191 | 219 | 650 | 282 | 142 | 172 | 470 | −205 | −42 |
| Mean | 314.86 | | 140.7 | 509.4 | 231.11 | | 114.7 | 434.9 | −83.75 | −27 |

where
Mean means Mean,
Mean diff. means Mean difference

The analysis of the results shown in Table 23 makes it possible to see the great difference in behavior between the bulls and to recreate the classification into group N bulls, with good libido (Bulls 1, 2, 3, 5 and 9), and group L bulls, with low libido (Bulls 4, 6, 7, 8 and 10), even if the difference between animals 8 and 9 is small.

For all bulls, except bull 6, administering composition C3 according to the invention reduces the reaction time in proportions ranging from 11% (for bull 1) to 79% (for bull 5). Only bull 6, belonging to the group of individuals with low libido, was penalized by 15% by the treatment.

For these bulls, it is also observed that the minimum and maximum reaction times are reduced by the treatment.

Table 24 below shows the results obtained in terms of ejaculation time for each bull, depending on the presence or absence of the treatment.

Each mean is calculated from 4 ejaculates (schedule 2*1) collected during the 4 weeks of the experimental period.

TABLE 24
Illustration of the individual “bull” effect and the absence of a
“bull/treatment” interaction on the ejaculation time (Tejac), in seconds,
on site 3 (composition C3)

| | Controls | | | | Treated | | | | | |
|---|---|---|---|---|---|---|---|---|---|---|
| | | Std. | | | | Std. | | | Mean | Mean |
| Tejac | Mean | dev. | Min. | Max. | Mean | dev. | Min. | Max. | diff. | diff. % |
| bull 1 | 755 | 179 | 520 | 954 | 857 | 68 | 779 | 936 | 102 | 14 |
| bull 2 | 1038 | 101 | 928 | 1157 | 750 | 77 | 670 | 820 | −288 | −28 |
| bull 3 | 927 | 106 | 802 | 1039 | 658 | 120 | 562 | 833 | −269 | −29 |
| bull 4 | 1600 | 486 | 1066 | 2030 | 1068 | 255 | 780 | 1397 | −532 | −33 |
| bull 5 | 1011 | 98 | 880 | 1114 | 636 | 58 | 565 | 685 | −375 | −37 |
| bull 6 | 1340 | 603 | 641 | 2010 | 1742 | 290 | 1510 | 2160 | 402 | 30 |
| bull 7 | 1515 | 583 | 1070 | 2344 | 1556 | 260 | 1222 | 1824 | 41 | 3 |
| bull 8 | 1054 | 126 | 890 | 1192 | 736 | 155 | 600 | 957 | −318 | −30 |
| bull 9 | 964 | 177 | 800 | 1155 | 672 | 57 | 619 | 736 | −292 | −30 |
| bull 10 | 1180 | 182 | 950 | 1413 | 1170 | 241 | 863 | 1432 | −10 | −1 |
| Mean | 1138.4 | | 854.7 | 1440.8 | 984.5 | | 817 | 1178 | −153.9 | −14 |

where
Mean means Mean,
Mean diff. means Mean difference

Taking the 10 bulls tested as whole, the average time reduction induced by the treatment with composition C3 according to the invention is 154 seconds per bull, i.e. 14% of the mean ejaculation time of the controls.

For six bulls (bulls 2, 3, 4, 5, 8 and 9), their ejaculation time decreases with the treatment, by a value between 28% and 37%. Two bulls (bulls 7 and 10) showed no noticeable improvement, and the ejaculation time decreased for two others (bulls 1 and 6). However, these behavioral differences are not enough to identify a “bull/treatment” interaction.

For 8 out of 10 individuals, the maximum ejaculation time decreases thanks to composition C3. Bull 10 is not affected by the treatment, and this parameter increases for bull 6. Taking the 10 bulls as whole, it is however observed that the treatment induces a decrease in the maximum average ejaculation time of 262 seconds, i.e. a reduction of 18% compared to the controls.

The above results clearly show a highly significant individual “bull” effect, and an absence of a “bull/treatment” interaction. The same results were obtained on all the other test sites for all the compositions tested. This tends to prove that the beneficial effect of the treatment on the reaction time and ejaculation time is entirely due to the tested molecules according to the invention.

The above description clearly illustrates that through its various features and their advantages the present invention realizes the objectives it set itself. In particular, it provides compositions that induce a significant improvement in the reproductive function in a bull. In particular, experiments carried out have demonstrated a significant improvement in the libido of bulls; using compositions according to the invention enables the reaction time to be reduced by about 30% and the ejaculation time by about 20%. Such time reductions will have significant positive impacts on the organization of semen collection, production costs and the safety of bull herders in contact with animals.

### BIBLIOGRAPHY

Kumar K R, Archunan G, Jeyaraman R & Narasimhan S (2000) Chemical characterization of bovine urine with special reference to oestrus, Vet Res Commun, 24 (7), 445-454.
Sankar R & Archunan G (2004) Flehmen response in bull: role of vaginal mucus and other body fluids of bovine with special reference to estrus, Behav Processes, 67 (1), 81-86.
Sankar R & Archunan G (2008) Identification of putative pheromones in bovine (Bos taurus) faeces in relation to estrus detection, Anim Reprod Sci, 103 (1-2), 149-153.

1.-19. (canceled)

20. A method to improve the reproductive function of a bull which comprises administering to the bull a composition comprising at least one volatile molecule specific to the estrus of cows which is selected from the group consisting of coumarin, squalene, 6-amino undecane, 2-butanone, 9-octadecenoic acid, 1,2-dichloroethylene and mixtures thereof.

21. The method of claim 1 wherein improving reproductive function includes improving the bull's libido.

22. The method of claim 1 wherein improving reproductive function includes improving the production of semen by the bull.

23. The method of claim 1 wherein the composition is in the form of a solution comprising at least glycerol as a solvent of the volatile molecules.

24. The method of claim 1 wherein the total concentration of the at least one molecule specific to estrus in the composition is between 2.5 pg/mL and 500 pg/mL.

25. The method of claim 1 wherein the total concentration of the at least one molecule specific to estrus in the composition is between 100 pg/mL and 500 pg/mL.

26. The method of claim 1 wherein the concentration of the at least one molecule specific to the estrus in the composition is at least equal to 25 pg/mL, +/−5%.

27. The method of claim 1 wherein the composition comprises a mixture of two or more of volatile molecules selected from the group consisting of coumarin, squalene, 6-amino undecane, 2-butanone, 9-octadecenoic acid, and 1,2-dichloroethylene.

28. The method of claim 1 wherein the composition comprises a mixture of squalene and 1,2-dichloroethylene.

29. The method of claim 1 wherein the composition is administered by inhalation.

30. The method of claim 29 wherein the composition is kept at the periphery of the bull's nostrils so that the bull can inhale said composition.

31. The method of claim 20 wherein the composition is sprayed into the bull's nostrils.

32. The method of claim 1 further comprising the bull serving a female or collection of semen from the bull directly after said composition has been administering to the bull.

33. The method of claim 1 wherein the composition is administered to the bull before each semen collection or before each covering.

34. A composition for administration to a bull to improve its reproductive function which comprises at least one volatile molecule specific to the estrus of cows selected from the group consisting of coumarin, squalene, 6-amino undecane, 2-butanone, 9-octadecenoic acid, 1,2-dichloroethylene or mixtures thereof.

35. The composition of claim 34 wherein the composition contains glycerol as a solvent of the at least one volatile molecule.

36. The composition of claim 34 wherein the composition comprises the excipients necessary to enable said composition to be sprayed.

37. The composition of claim 34 wherein the total concentration of the at least one volatile molecule specific to the estrus of cows is between 2.5 pg/mL and 500 pg/mL.

38. The composition of claim 34 wherein the concentration of the at least one volatile molecule specific to the estrus of cows is at least equal to 25 pg/mL, +/−5%.

39. The composition of claim 34 wherein the at least one volatile molecule is a mixture of squalene and 1,2-dichloroethylene.
